(19) United States
(12) Patent Application Publication
(10) Pub. No.: US 2006/0128248 A1
(43) Pub. Date: Jun. 15, 2006

(54) Nonwoven sanitizing wipe including an anionic binder formulation

(75) Inventor: Dianne Ellis, Cary, NC (US)

Correspondence Address: WOOD, PHILLIPS, KATZ, CLARK & MORTIMER, 500 W. MADISON STREET, SUITE 3800, CHICAGO, IL, 60661, US

(73) Assignee: PGI Polymer, Inc.

(21) Appl. No.: 11/280,504

(22) Filed: Nov. 16, 2005

Related U.S. Application Data
(60) Provisional application No. 60/628,180, filed on Nov. 16, 2004.

(51) Int. Cl. B32B 27/02; D04H 1/46; D04H 3/10

(52) U.S. Cl. 442/408; 442/164; 442/170; 442/171

(57) ABSTRACT

A nonwoven wipe, a wiping system and method for using a wipe are provided that offer superior sanitization capabilities over prolonged periods of use. The novel combination of nonwoven substrate having wettable polypropylene and an anionic binder formulation results in a wipe that exhibits washable durability and is capable interacting with a chlorine sanitizer, either disposed in the wipe or in ancillary chlorine sanitizing solution, for prolonged periods of use without significantly depleting the chlorine content of the wipe or solution.

### CROSS-REFERENCE TO RELATED APPLICATION

This application claims priority of U.S. Provisional Application Ser. No. 60/628,180, filed Nov. 16, 2004.

### FIELD OF THE INVENTION

The present invention generally relates to a nonwoven sanitizing wipe, and more particularly to a nonwoven sanitizing wipe formed of wettable polypropylene and an anionic binder.

### BACKGROUND OF THE INVENTION

Over the years, the use of disposable substrates in cleaning applications has been well established. Suitable substrates have included sponges, woven and nonwoven fabrics, and various combinations thereof. Further, such substrates have been impregnated with cleaning agents such as disinfectants, solvents, anti-microbials, detergents and other chaotropes. The resulting cleaning products fabricated from such impregnated substrates have found acceptance with the general public as a convenient and practical means for the cleaning of surfaces. In particular, such constructs have been successful in the consumer wipes markets as hard surface wipes.

Nonwoven fabrics are used in a wide variety of wipe applications where the engineered qualities of the fabrics can be advantageously employed. The use of selected thermoplastic polymers in the construction of the fibrous fabric component, selected treatment of the fibrous component (either while in fibrous form or in an integrated structure), and selected use of various mechanisms by which the fibrous component is integrated into a useful fabric, are typical variables by which to adjust and alter the performance of the resultant nonwoven fabric.

Nonwoven hard surface wipes fabricated for the food service or hospitality industry, ordinarily comprise a fibrous blend of 70/30 PET/rayon, as well as a binder formulation. Such wipes have gained in popularity over the years versus sponges and woven terry towel, due to the benefits that come along with utilizing a limited or single use nonwoven wipe. Reusable damp sponges or woven terry cloth towels are thought to be more susceptible to bacterial build up as they are left standing between uses. It is also thought that repeatedly using a standing damp towel or sponge to wipe down a food preparation surface may actually be re-introducing harmful bacteria to the food preparation area.

As the use of nonwoven wipes in the foodservice industry has increased, binder formulations have been developed that benefit the durability of the wipe, in particular provide wash durability for the purpose of developing multi-use, re-usable wipes. In spite of these developments, it has been found that certain binder formulations have a detrimental effect on maintaining the effectiveness of the sanitizing solution. In light of such findings, there remains a need for a nonwoven hard surface wipe for the foodservice and hospitality industry that utilizes a binder formulation that can be used in conjunction with a chlorinated sanitizing solution without significantly depleting the level of effectiveness of the solution over a given period of time.

### SUMMARY OF THE INVENTION

The present invention defines a non-woven wipe, as system including the nonwoven wipe and a method for using the nonwoven wipe. The wipe includes a nonwoven substrate having at least 20% wettable polypropylene and anionic binder formulation. The binder allows provides for a durable/washable wipe and the anionic binder provides the capability to interact with a chlorinated cleaning solution for a prolonged period of time, in excess of 4 hours, without significantly depleting the chlorine concentration in the cleaning solution. Thus, the user of the wipe can be assured that the wipe is providing continuous sanitization throughout a prolonged period of use.

In one embodiment of the invention a wipe article is defined that includes a nonwoven substrate that includes at least 20 percent by weight wettable polypropylene and an anionic binder formulation disposed within the substrate. In specific embodiments, the substrate of the wipe may be hydroentangled and may comprise about 25 percent by weight and about 30 percent by weight wettable polypropylene. Additionally, the anionic binder formulation may include an anionic acrylic binder, an anionic surfactant and/or a non-ionic anti-foam. For example, in one embodiment the anionic binder formulation may include at least 0.2 percent by weight anionic acrylic binder, in another embodiment the anionic binder formulation may include at least 0.05 percent by weight anionic surfactant and in yet another embodiment the anionic binder formulation may include at least 0.001 percent by weight non-ionic anti-foam.

Additionally, the wipe article may include a chlorine additive impregnated in the wipe article. In this embodiment, the anionic binder formulation will provide for the wipe to maintain at least 88 percent of the initial chlorine additive after a 4 hour period of use.

The invention is also defined by a system for sanitizing a surface work, such as a foodservice work area or the like. The system includes a nonwoven wipe that includes a nonwoven substrate that is at least 20 percent by weight wettable polypropylene and an anionic binder formulation disposed within the substrate. Additionally, the system includes a chlorinated cleaning solution that maintains at least 88 percent of an initial chlorine concentration after a 4 hour period of use with the nonwoven wipe. In specific embodiments, the substrate of the wipe may be hydroentangled and may comprise about 25 percent by weight and about 30 percent by weight wettable polypropylene. Additionally, the anionic binder formulation of the wipe may include an anionic acrylic binder, an anionic surfactant and/or a non-ionic anti-foam. For example, in one embodiment the anionic binder formulation may include at least 0.2 percent by weight anionic acrylic binder, in another embodiment the anionic binder formulation may include at least 0.05 percent by weight anionic surfactant and in yet another embodiment the anionic binder formulation may include at least 0.001 percent by weight non-ionic anti-foam.

The invention is also defined by a method for sanitizing a surface area, such as a foodservice work area or the like. The method includes the steps of providing for a nonwoven wipe that includes a nonwoven substrate that is at least 20 percent by weight wettable polypropylene and an anionic binder formulation disposed within the substrate, providing for a chlorinated cleaning solution and sanitizing with the nonwoven wipe and the chlorinated cleaning solution for a period of at least four hours, such that the chlorinated cleaning solution maintains at least 88 percent of the initial chlorine concentration after a four hour period of use.

Thus, the present invention provides for a nonwoven wipe, a wiping system and method for using a wipe that provide superior sanitization capabilities over prolonged periods of use. The novel combination of nonwoven substrate having wettable polypropylene and an anionic binder formulation results in a wipe that exhibits washable durability and is capable interacting with a chlorine sanitizer, either disposed in the wipe or in ancillary chlorine sanitizing solution, for prolonged periods of use without significantly depleting the chlorine content of the wipe or solution. Such wipe insures that adequate sanitization is occurring over the useful life of the nonwoven wipe.

### BRIEF DESCRIPTION OF THE DRAWINGS

FIG. 1 is a schematic drawing of a hydroentangling apparatus for forming nonwoven fabrics in accordance with the present invention.

### DETAILED DESCRIPTIONS OF THE PREFERRED EMBODIMENTS

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

The present invention is directed to nonwoven wipes, systems for sanitizing using the nonwoven wipes and methods for sanitizing using the nonwoven wipes. The wipe combines a nonwoven substrate having at least 20% wettable polypropylene with an anionic binder formulation. Such a wipe acts to deter depletion of chlorine in a chlorinated cleaning solution or in instances in which the wipe is impregnated with a chlorinated additive. Therefore, the wipe insures that adequate sanitization is occurring over the useful life of the nonwoven wipe.

Formation of the wiping substrate is such that the wipe is comprised of at least about 20% wettable polypropylene, more preferably about 25% wettable polypropylene, and most preferably about 30% wettable polypropylene.

According to the invention, the binder formulation is comprised of an anionic acrylic binder, an anionic surfactant, and/or non-ionic anti-foam. Suitable anionic acrylic binders include, but are not limited to ABX30 and 25-4260, made commercially available by Celanese Corporation of Dallas, Tex. and Hycar 2671, which is a commercial product provided by Noveon Inc. of Cleveland Ohio. Preferably, the anionic formulation is about 0.2% to about 20% self-crosslinking acrylic anionic binder, preferably about 0.05% to about 5.0% anionic surfactant, and preferably about 0.001% to about 1.0% non-ionic anti-foam. Embodiments of the sanitizing wipes are provided in the examples below, however the following examples are not meant to limit the scope of the present invention.

The nonwoven substrate may comprise any suitable matrix of fibers or filaments that are typically consolidated into a nonwoven web. For example, staple length fibers, continuous filaments or blends of fibers and/or filaments having the same or different compositions may be used to form the substrate. The fibers and/or filaments may be selected from natural or synthetic composition and they may be homogeneous or mixed fiber/filament length. Suitable natural fibers include, but are not limited to, cotton, wood pulp, viscose rayon and the like. Synthetic fibers, which may be blended in whole or part, include, but are not limited to, thermoplastic and thermoset polymers.

Thermoplastic polymers suitable for blending with dispersant thermoplastic resins include polyolefins, polyamides and polyesters. The. thermoplastic polymers may be further selected from homopolymers, copolymers, conjugates and other derivatives including those thermoplastic polymers having incorporated melt additives or surface-active agents. Staple lengths are selected in the range of about 0.25 inch to about 10 inches, the range of about 1 to about 3 inches being preferred and the fiber denier selected in the range of about 1 to about 22, the range of about 1.2 to about 6 denier being preferred for general applications. The profile of the fiber and/or filament is not a limitation to the applicability of the present invention.

It is also within the purview of the present invention that the wipe comprise additional fabric layers so as to form a laminate construct. The additional layers may include, but are not limited to, fabrics comprised of natural, synthetic fibers, or a combination thereof. Suitable natural fibers include, but are not limited to, cotton, wood pulp and viscose rayon. Synthetic fibers, which may be blended in whole or part, include thermoplastic and thermoset polymers. The thermoplastic polymers may be further selected from homopolymers, copolymers, conjugates and other derivatives including those thermoplastic polymers having incorporated melt additives or surface-active agents. Filamentary nanofiber fabrics as represented by U.S. Pat. No. 6,114,017, entitled “Micro-Denier Nonwoven Materials Made Using Modular Die Units”, issued Sept. 5, 2000, in the name of inventors Fabbricante et al; U.S. Pat. No. 6,269,513, entitled “Wipe Pads With Superior Solids Removal Ability Using Sub-Micron Filaments”, issued Aug. 7, 2001, in the name of inventor Torobin; and U.S. Pat. No. 6,315,806, entitled, “Method and apparatus for producing high efficiency fibrous media incorporating discontinuous sub-micron diameter fibers, and web media formed thereby”, issued Nov. 13, 2001, in the name of inventor Torobin, all of which are incorporated herein by reference as if set forth fully herein, may also be utilized in combination with the present invention to form a wipe article. Additionally, film layers may be added to form a laminate construct. Various film layers may include, cast films, extruded films, and reticulated films.

The wipe itself may include a chlorine additive or the wipe may be devoid of a chlorine additive. It is also been contemplated and within the scope of the present invention that the sanitation wipes optionally include an other additives or combination of additives, such as pigments, color stabilizers, softeners, fragrances, lotions, other performance or aesthetic enhancers and the like.

The invention may also be defined by a system for providing sanitization to a work area, such as a foodservice work area or the like. The system includes a nonwoven wipe that includes a nonwoven substrate that is at least 20 percent by weight wettable polypropylene and an anionic binder formulation disposed within the substrate and a chlorinated cleaning solution that maintains at least 88 percent of an initial chlorine concentration after a 4 hour period of use with the nonwoven wipe. The nonwoven substrate that forms the wipe will typically be formed of between about 25 percent by weight and about 30 percent by weight polypropylene. The anionic binder formulation may include at least 0.2 percent by weight anionic acrylic binder, at least 0.05 percent by weight anionic surfactant and at least 0.001 percent by weight non-ionic anti-foam.

Additionally, the invention may be defined by a method for sanitizing a surface area, such as a foodservice or hospitality work area. The method includes the steps of providing for a nonwoven wipe that includes a nonwoven substrate that is at least 20 percent by weight wettable polypropylene and an anionic binder formulation disposed within the substrate, providing for a chlorinated cleaning solution and sanitizing with the nonwoven wipe and the chlorinated cleaning solution for a period of at least four hours, wherein the chlorinated cleaning solution maintains at least 88 percent of the initial chlorine concentration after a four hour period of use.

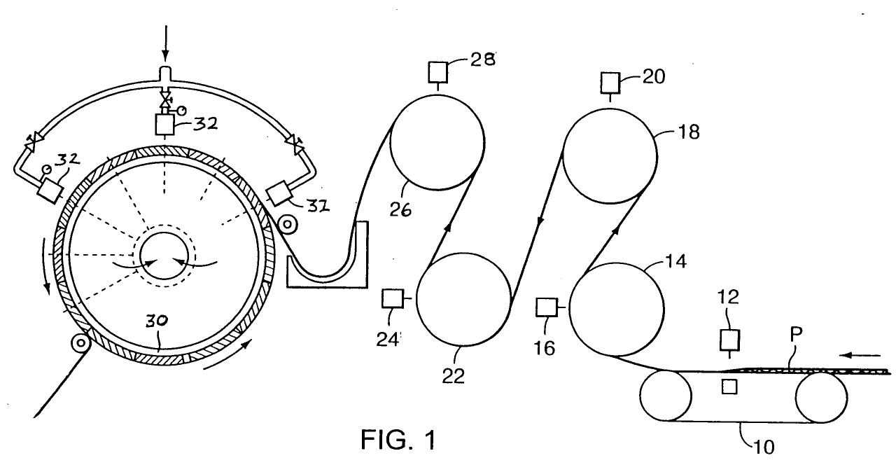

With reference to FIG. 1, therein is illustrated a schematic diagram of an apparatus for making the wipe of the present invention, in accordance with an embodiment of the present invention. As depicted the wipe is formed by hydroentangling a nonwoven fabric. the methods of forming the wipe are also possible and within the inventive concepts herein disclosed. The fabric that comprises the wipe is formed from a fibrous matrix, which typically comprises staple length fibers, but may comprise substantially continuous filaments. The fibrous matrix is typically carded and cross-lapped to form a fibrous batt. In one embodiment, the fibrous batt comprises 100% cross-lap fibers, that is, all of the fibers of the web have been formed by cross-lapping a carded web so that the fibers are oriented at an angle relative to the machine direction of the resultant web. U.S. Pat. No. 5,475,903, entitled, “Composite Nonwoven Fabric And Method”, issued on Dec. 19, 1995, in the name of inventor Collins; hereby incorporated by reference, illustrates an exemplary web drafting apparatus. Alternately, the fabric of the present invention may comprise an optional support layer or scrim, wherein the fibrous web can be placed face-to-face with the support layer and hydroentangled to form precursor web P. It is within the purview of the present invention that a scrim can be interposed in the formation of the precursor nonwoven web. The purpose of the scrim is to reduce the extensibility of the resultant three-dimensional imaged nonwoven fabric, thus reducing the possibility of three-dimensional image distortion and further enhancing fabric durability. Suitable scrims include unidirectional monofilament, bi-directional monofilament, expanded films, and thermoplastic spunbond.

FIG. 1 illustrates a hydroentangling apparatus for forming nonwoven fabrics in accordance with the present invention. The apparatus includes a foraminous-forming surface in the form of belt 10 upon which the fibrous web P is positioned for entangling by entangling manifold 12. Further entanglement of the web is effected on the foraminous forming surface of a drum 14 by entanglement manifold 16, with the web subsequently passed over successive foraminous drums 18, 22, 26 for successive entangling treatment by entangling manifolds 20, 24, 28.

The entangling apparatus of FIG. 1 may further include a foraminous image forming surface, such as a three-dimensional imaging drum 30 comprising a three-dimensional image transfer device for effecting imaging of the now-entangled precursor web. The image transfer device includes a moveable imaging surface which moves relative to a plurality of entangling manifolds 32 which act in cooperation with three-dimensional elements defined by the imaging surface of the image transfer device to effect imaging and patterning of the fabric being formed.

Embodiments of the sanitizing wipes are provided in the examples below, however the following examples are not meant to limit the scope of the present invention. The Example 1 illustrates a substrate without an anionic binder formulation. Without a binder formulation the substrate does not readily deplete the chlorine concentration in a solution after a prolonged period of use. Examples 2-4 illustrate a substrate including an anionic binder system. While a binder system will typically cause the chlorine concentration to rapidly deplete over prolonged use, use of the anionic binder system of the present invention allows for the chlorine concentration to remain relatively and effectually high over a prolonged period of use.

### EXAMPLE 1

A fibrous hydroentangled substrate comprising 30% T-130 wettable polypropylene/30% Tencel® (Lenzing Fibers, New York, N.Y.)/40% 310P polyester. The substrate was immersed in a chlorinated solution with an initial chlorine reading of 108.2 ppm (parts per million). The substrate was left in the chlorinated solution for four hours and the solution was tested again for effectiveness. After four hours, the solution had an effectual chlorine reading of 102.9 ppm (maintains approximately 95% chlorine effectiveness).

### EXAMPLE 2

A fibrous hydroentangled substrate comprising 30% T-130 wettable polypropylene/30% Tencel®/40% 310P polyester and an anionic binder system (1% Hycar 2671 anionic acrylic binder (Noveon Inc. of Cleveland Ohio), 0.1% Wet Doss anionic surfactant (FibroChem Inc. of Dalton, Ga.), 0.0125% non-ionic antifoam). The substrate was immersed in a chlorinated solution with an initial effectual chlorine reading of 107.4 ppm. The substrate was left in the chlorinated solution for four hours and the solution was tested again for effectiveness. After four hours, the solution had an effectual chlorine reading of 100.5 ppm. (maintains approximately 93.5% chlorine effectiveness).

### EXAMPLE 3

A fibrous hydroentangled substrate comprising 30% T-130 wettable polypropylene/30% Tencel®/40% 310P polyester and an anionic binder system (1% 25-4260 anionic acrylic binder (Celanese Corporation of Dallas, Tex.), 0.1% Wet Doss anionic surfactant, 0.0125% non-ionic antifoam) was immersed in a chlorinated solution with an initial effectual chlorine reading of 108 ppm. The substrate was left in the chlorinated solution for four hours and the solution was tested again for effectiveness. After four hours, the solution had an effectual chlorine reading of 100.1 ppm (maintains approximately 92.5% chlorine effectiveness).

### EXAMPLE 4

A fibrous hydroentangled substrate comprising 30% T-130 wettable polypropylene/30% Tencel®/40% 310P polyester and an anionic binder system (2% Vinamul ABX-30 anionic acrylic binder (Celanese Corporation of Dallas, Tex.), 0.6% Wet Doss anionic surfactant, 0.0125% non-ionic anti-foam) was immersed in a chlorinated solution with an initial effectual chlorine reading of 107.1 ppm. The substrate was left in the chlorinated solution for four hours and the solution was tested again for effectiveness. After four hours, the solution had an effectual reading of 95.9 ppm (maintain approximately 89.5% chlorine effectiveness).

Table 1 illustrates the sanitizing wipes disclosed herein, as well as comparative performance data for 100% cotton woven towels and additional nonwoven substrates that show a depletion of the chlorinated sanitation solution to levels that compromise the effectiveness of the sanitation process.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

TABLE 1

| | | | | | PPM Available | |
|---|---|---|---|---|---|---|
| | | | | | Chlorine | |
| SAMPLE | CODE | TECHNOLOGY | FIBER BLEND | BINDER SYSTEM | 0 HR. | 4 HR. |
| 0 | CONTROL | N/A | N/A | N/A | 108.8 | 105.7 |
| 1 | DE205 Base | Spunlace | 30% T-130 Wettable | N/A | 108.2 | 102.9 |
| | | | PP/30% Tencil/40% 310P PET | | | |
| 2 | DE205A | Spunlace | 30% T-130 Wettable | 1% Hycar 2671, 01.% Wet Doss, | 107.4 | 100.5 |
| | Binder | | PP/30% Tencil/40% 310P PET | 0.0125% Antifoam PGI | | |
| 3 | DE 205 B | Spunlace | 30% T-130 Wettable | 1% 25-4260, 0.1% Wet Doss, | 108 | 100.1 |
| | Binder | | PP/30% Tencil/40% 310P PET | 0.0125% Antifoam PGI | | |
| 4 | DE 205 C | Spunlace | 30% T-130 Wettable | 2% Vinamul ABX-30, 0.6% Wet | 107.1 | 95.9 |
| | Binder | | PP/30% Tencil/40% 310P PET | Doss, 0.0125% Anifoam PGI | | |
| 5 | DE 205 D | Spunlace | 30% T-130 Wettable | 0.5% Hycar 2671, 0.6% Wet Doss, | 107.4 | 101.6 |
| | Binder | | PP/30% Tencil/40% 310P PET | 0.125% Antifoam PGI | | |
| 6 | Bar Towell, | Woven | 100% Cotton | N/A | 101.3 | 67 |
| | unwashed | | | | | |
| 7 | Bar Towel, 1 | Woven | 100% Cotton | N/A | 104.5 | 69.3 |
| | wash cycle | | | | | |
| 8 | Bar Towel, 3 | Woven | 100% Cotton | N/A | 106.9 | 81.6 |
| | wash cycles | | | | | |
| 9 | Bar Towel, 3 | Woven | 100% Cotton | N/A | 107.3 | 82.7 |
| | wash cycles | | | | | |
| 10 | Bar Towel, 6 | Woven | 100% Cotton | N/A | 106 | 82.9 |
| | wash cycles | | | | | |
| 11 | Bar Towel, 6 | Woven | 100% Cotton | N/A | 106.6 | 81 |
| | wash cycles | | | | | |
| 12 | Huck Towel, | Woven | 100% Cotton | N/A | 110.6 | 21 |
| | unwashed | | | | | |
| 13 | Huck Towel, 1 | Woven | 100% Cotton | N/A | 105 | 37.5 |
| | wash cycle | | | | | |
| 14 | Huck Towel, 3 | Woven | 100% Cotton | N/A | 107 | 44.2 |
| | wash cycles | | | | | |
| 15 | Huck Towel, 3 | Woven | 100% Cotton | N/A | 106.5 | 37.9 |
| | wash cycles | | | | | |
| 16 | Huck Towel, 6 | Woven | 100% Cotton | N/A | 107.3 | 35.5 |
| | wash cycles | | | | | |
| 17 | Huck Towel, 6 | Woven | 100% Cotton | N/A | 107.1 | 27.7 |
| | wash cycles | | | | | |
| Previous | Standard | Spunlace | 35% rayon/35% Tencel H21 | 0.0625% DAP, 0.15% Wet Doss, | 103 | 74 |
| testing | Foodservice | | 5968; 30% PET | 0.025% Antifoam PGI, 6.5% 25- | | |
| | (code 5793) | | | 4260, 1% Ultrafast 3000 NFP, | | |
| | | | | 0.84% Microban | | |
| Previous | Standard | Spunlace | 35% rayon/35% Tencel H215 | 1.25% Ultrafast 3000 NFP, 0.5% | 25 | 17 |
| testing | SmartBand | | | Mirataine H2C-HA, 5.3% 25-212A, | | |
| | case good | | | 5.5% Glo-Tex 727, 0.0125% | | |
| | | | | Antifoam PGI, 7.5 Compad Soil | | |
| | | | | Release, 0.7% Microban | | |
| Previous | Wet Wipe | Adhesive | 75% 8195/25% PET | 0.375% Antifoam 1510, 62.5% E- | 110 | 94 |
| testing | (code 5408 | Bond | | 646, 0.75% Wetlaid SR Special, 1% | | |
| | | | | Astron Red 2BRS, 0.19% | | |
| | | | | Ammonium Chloride | | |

All towels cut to same size (14: × 17″).
Cotton towels purchased from Image Textiles.

| | | | |
|---|---|---|---|
| BINDERS: | | CHEMICALS: | |
| ABX-30 | Anionic self-crosslinking acrylic emulsion | Astro West Doss | Anionic surfactant |
| Hycar 2671 | Anionic acrylic | PGI antifoam | Nonionic antifoam |
| 25-4260 | Anionic self-crosslinking acrylic copolymer emulsion | | |

What is claimed is:

1. A wipe article, the article comprising:
a nonwoven substrate comprising at least 20 percent by weight wettable polypropylene; and
an anionic binder formulation disposed within the substrate.

2. The article of claim 1, further comprising a chlorine additive impregnated in the wipe article.

3. The article of claim 2, wherein the wipe maintains at least 88 percent of the chlorine additive after a 4 hour period of use.

4. The article of claim 1, wherein the nonwoven substrate comprises between about 25 percent by weight and about 30 percent by weight polypropylene.

5. The article of claim 1, wherein the nonwoven substrate is further defined as a hydroentangled nonwoven substrate.

6. The article of claim 1, wherein the anionic binder formulation comprises at least 0.2 percent by weight anionic acrylic binder.

7. The article of claim 1, wherein the anionic binder formulation comprises at least 0.05 percent by weight anionic surfactant.

8. The article of claim 1, wherein the anionic binder formulation comprises at least 0.001 percent by weight non-ionic anti-foam.

9. A wipe article, the article comprising:
a nonwoven substrate comprising at least 20 percent by weight wettable polypropylene;
an anionic binder formulation disposed within the substrate; and
a chlorinated additive disposed within the substrate.

10. The article of claim 9, wherein the nonwoven substrate comprises between about 25 percent by weight and about 30 percent by weight polypropylene.

11. The article of claim 9, wherein the anionic binder formulation comprises at least 0.2 percent by weight anionic acrylic binder.

12. The article of claim 9, wherein the anionic binder formulation comprises at least 0.05 percent by weight anionic surfactant.

13. The article of claim 9, wherein the anionic binder formulation comprises at least 0.001 percent by weight non-ionic anti-foam

14. A system for sanitizing a surface area, the system comprising:
a nonwoven wipe that includes a nonwoven substrate that is at least 20 percent by weight wettable polypropylene and an anionic binder formulation disposed within the substrate; and
a chlorinated cleaning solution that maintains at least 88 percent of an initial chlorine concentration after a 4 hour period of use with the nonwoven wipe.

15. The system of claim 14, wherein the nonwoven substrate comprises between about 25 percent by weight and about 30 percent by weight polypropylene.

16. The system of claim 14, wherein the nonwoven substrate is further defined as a hydroentangled nonwoven substrate.

17. The system of claim 14, wherein the anionic binder formulation comprises at least 0.2 percent by weight anionic acrylic binder.

18. The system of claim 14, wherein the anionic binder formulation comprises at least 0.05 percent by weight anionic surfactant.

19. The system of claim 14, wherein the anionic binder formulation comprises at least 0.001 percent by weight non-ionic anti-foam.

20. A method for sanitizing a surface area, the method comprising the steps of:
providing for a nonwoven wipe that includes a nonwoven substrate that is at least 20 percent by weight wettable polypropylene and an anionic binder formulation disposed within the substrate;
providing for a chlorinated cleaning solution; and
sanitizing with the nonwoven wipe and the chlorinated cleaning solution for a period of at least four hours, wherein the chlorinated cleaning solution maintains at least 88 percent of the initial chlorine concentration after a four hour period of use.
